(19) United States
(12) Patent Application Publication
(10) Pub. No.: US 2025/0319016 A1
(43) Pub. Date: Oct. 16, 2025

(54) COMPOSITION COMPRISING A HEMP DERIVATIVE AND A PICKERING EMULSION , PREPARATION AND USES

(71) Applicant: AHAITUKI SAS, ARZON (FR)

(72) Inventor: Louis-François OMNES, PARIS (FR)

(21) Appl. No.: 19/178,303

(22) Filed: Apr. 14, 2025

(30) Foreign Application Priority Data
Apr. 15, 2024 (FR) FR2403896

(51) Int. Cl. A61K 8/9789; A61K 8/06; A61K 8/25; A61K 8/34; A61K 8/368; A61K 8/49; A61K 8/92; A61Q 19/00

(52) U.S. Cl. CPC A61K 8/9789; A61K 8/062; A61K 8/25; A61K 8/347; A61K 8/368; A61K 8/498; A61K 8/922; A61Q 19/00; A61K 2800/782

(57) ABSTRACT

A composition includes a solution containing at least one hemp derivative and a Pickering O/W emulsion, the hemp derivative being selected from hemp flower oily macerates, cannabinoids, terpenes and flavonoids and the Pickering O/W emulsion containing an aqueous phase, a vegetable oil phase and an organomodified phyllosilicate, process for preparing the composition and use as a soothing, relaxing, anti-stress agent for the skin.

### CROSS-REFERENCE TO RELATED APPLICATIONS

This application claims priority to French Patent Application No. FR2403896, filed Apr. 15, 2024, the entire content of which is incorporated herein by reference in its entirety.

### FIELD

The present invention relates to the field of compositions based on hemp derivatives and in particular based on hemp flower oil(s). More specifically, the invention concerns a composition comprising at least one oily macerate of hemp flowers and its use in cosmetics for application to the skin, in particular as an anti-stress, soothing, relaxing agent, for the skin.

### BACKGROUND

The skin is the human body's main protective barrier against external aggressors such as atmospheric pollution, climatic variations and UV radiation.

The skin is made up of three layers: epidermis, dermis and hypodermis. The epidermis, which is in contact with the surface layer, is particularly concerned by interactions with the external environment. The epidermis is covered by a hydrolipidic film and comprises four layers: the stratum corneum, the stratum granulosum, the stratum spinosum and the stratum basale.

Patent application WO2016/123475 describes a composition comprising a Cannabis oil extract and vitamin E. In the paragraph devoted to the prior art, the authors highlight the viscous nature of Cannabis oils and the difficulties arising therefrom, particularly when they are to be used by vaporization.

The viscosity of cannabis oils is therefore a parameter to be taken into consideration when developing a composition with a particular galenic form suitable for application to the skin.

### SUMMARY

The inventor has shown that a solution to the technical problem of obtaining new soothing cosmetic compositions can be provided by means of a composition comprising a solution containing at least one hemp derivative and a O/W Pickering emulsion (i.e. an oil dispersed in water), said hemp derivative being chosen from hemp flower oily macerates, cannabinoids, terpenes and flavonoids, and the O/W Pickering emulsion containing an aqueous phase, a vegetable oil phase and an organomodified phyllosilicate. In particular, the composition according to an aspect of the invention comprises at least one oily macerate of hemp flowers and a Pickering emulsion.

Hemp or Cannabis is a flowering herbaceous plant in the Cannabinaceae family. This annual plant, native to Central and South Asia, has several phenotypes that can be described as subspecies and varieties.

Hemp contains over 540 active ingredients identified today, belonging to various chemical classes, including cannabinoids, terpenes and flavonoids (1).

The various parts of the plant are exploited for a wide range of industrial uses. Feminized flowers are mainly used in the medical, wellness and cosmetics sectors, in particular to extract two molecules from the cannabinoid family: Δ-9-tetrahydrocannabinol, also known simply as “tetrahydrocannabinol” or THC, and Cannabidiol or CBD.

The scientific community has now established the existence of an “entourage effect” for Cannabis, i.e. the interaction and synergy of compounds of interest contained in the plant, resulting in products with an optimized benefit-risk profile (2,3). New attention is now being paid to new molecules from the terpene and flavonoid families, as well as other cannabinoids such as Cannabigerol or CBG.

The “entourage effect” calls into question the relevance of the historical development strategies of the above-mentioned hemp industries, exploiting almost exclusively one or two molecules, THC and CBD, used alone or in combination.

Surprisingly and beneficially, the inventor has identified a particular composition with beneficial cosmetic effects when applied to the skin, more particularly as a soothing, relaxing, anti-stress agent. It will be appreciated that this enhancement is not observed when the individual components—namely the hemp flower oily macerate or the Pickering emulsion—are used alone, nor when conventional clays such as kaolin are employed. The inventor has further discovered that within specific ranges of oily macerate and organomodified phyllosilicate do these synergistic and unexpectedly beneficial results emerge

According to a first aspect, the present invention relates to a composition comprising a solution containing at least one hemp derivative and a O/W Pickering emulsion, said hemp derivative being selected from hemp flower oily macerates, cannabinoids, terpenes and flavonoids and the O/W Pickering emulsion containing an aqueous phase, a vegetable oil phase and an organomodified phyllosilicate.

According to a second aspect, the present invention concerns a composition comprising a solution containing at least one hemp flower oily macerate and a O/W Pickering emulsion containing an aqueous phase, a vegetable oil phase and an organomodified phyllosilicate.

In an embodiment, the solution containing at least one oily macerate of hemp flowers is present in a content ranging from 0.1 to 25%, such as from 1% to 5% and beneficially 3.4% by weight relative to the total weight of the composition.

An aspect of the present invention also covers a process for preparing the composition according to the invention.

An aspect of the present invention is also aimed at the use of the composition according to an aspect of the invention as a soothing, relaxing, anti-stress agent for the skin.

An aspect of the present invention also covers a skin treatment process, comprising at least one step of applying the composition according to the invention to the skin.

These cosmetic compositions are also stable, non-irritating, non-toxic and non-allergenic to the skin.

Further aspects, benefits and properties of the present invention are presented in the following description and examples.

### DETAILED DESCRIPTION

### Definitions

In the present text, unless otherwise specified, percentages are expressed by weight of a reference composition.

In this text, intervals are defined in abbreviated form to avoid describing each and every value in the interval, but any suitable value in the interval can be chosen as the upper value, the lower value or the terminal values of the interval. For example, an interval from 0.1 to 1.0 represents the terminal values of 0.1 and 1.0, as well as the intermediate values of 0.2; 0.3; 0.4; 0.5; 0.6; 0.7; 0.8; 0.9 and all intermediate intervals within 0.1 to 1.0, such as 0.2 to 0.5; 0.2 to 0.8; 0.7 to 1.0 . . . . Unless otherwise specified, an interval defined as “between value A and value B” includes values A and B, and is therefore equivalent to an interval “from value A to value B”, the expression “at least” includes the value stated after it, e.g. “at least 5%” is to be understood as also including “5%”, the expression “a maximum of” includes the value stated after it, e.g. “a maximum of 5%” is to be understood as also including “5%”.

Furthermore, in the present text, measurable values, such as a quantity, are to be understood as including standard deviations which can be easily determined by the skilled person in the technical field of reference. In one or embodiments, these values are intended to include variations of ±5%.

In this text, the words “hemp” and “Cannabis” are used interchangeably.

Skin” refers to the epidermis of the face, body or scalp.

The compositions according to the present invention may be cosmetic, compositions. The cosmetic compositions are intended to be applied to healthy skin, by simple massage, to treat the epidermis. They comply with EC regulation 1223/2009.

### Hemp Oily Macerate(s)

The solution containing at least one hemp flower oily macerate usable according to an aspect of the invention is a solution comprising at least 50%, such as at least 75%, by weight of an oily macerate obtained by macerating hemp flowers in hemp seed oil.

In an embodiment, this macerate is obtained by macerating 500 to 1000 g of hemp flowers for 1 L of hemp seed oil.

The mass ratio of hemp flower to hemp seed oil is beneficially between 1/3 and 3/1, such as between 1/2 and 1/1.

Generally speaking, maceration, also known as “infusion”, lasts 3 to 4 weeks. It is up to the skilled worker to determine the duration of the maceration.

All hemp flowers can be used, provided, of course, that their use is authorized by the relevant national or transnational regulations.

In Europe, this is notably the case for strains with a high CBD content from the Common Catalogue of Varieties of Agricultural Plant Species, such as Kompolti, Carmagola, Perugina, Finola, or more recently Pain Killer, Midwest or Strawberry K. In other countries, notably the USA, other genetic strains of interest may be used, such as ACDC, Charlotte's web, Harlequin, Harle-Tsu, Ringo's gift.

Beneficially, the flowers used are manicured, that is the inflorescence has been harvested by hand in order to preserve the quality of the flower totum as much as possible.

A secondary macerate of hemp flowers in a vegetable oil other than hemp seed oil, can be added to this main macerate. Sunflower oil may be used as the vegetable oil other than hemp seed oil.

The addition of a second macerate in which other hemp flowers have been decarboxylated ensures a better “entourage effect” of the solution according to an aspect of the invention, that is ensuring that a greater variety of hemp flower compounds, with their specific cosmetic properties, are present in the solution.

Decarboxylation involves heating an oily macerate to convert the cannabinoids from their acid form to their active form, and can also be achieved by the action of UV light on the macerate. This process converts CBDA (Cannabidiol Acid) into CBD and THCA (Tetrahydrocannabinol Acid) into THC.

Beneficially, the volume ratio (v/v) between the oily macerate obtained by macerating hemp flowers in a hemp seed oil (main macerate) and the oily macerate obtained by macerating hemp flowers in a different oil (secondary macerate) is strictly greater than 1/1, such as 3/1.

In an embodiment, the solution containing at least one oily macerate of hemp flowers comprises at least CBD in a quantity ranging from 0.005% to 24.000%, such as from 0.010% to 5.000% by weight with respect to the total weight of the solution; CBDA in a quantity ranging from 0.005% to 24.000%, such as from 0.010% to 5.000% by weight with respect to the total weight of the solution; THC in an amount ranging from 0.001% to 30.000%, such as 0.010% to 1.000% by weight based on the total weight of the solution; CBG in an amount ranging from 0.001% to 22.000%, such as 0.010% to 3.000% by weight based on the total weight of the solution and CBGA in an amount ranging from 0.001% to 22.000%, such as 0.010% to 3.000% by weight based on the total weight of the solution.

It will be appreciated that these quantities will be adapted to national or transnational regulations in the country/countries where the operation is planned.

### Pickering Emulsions

Pickering emulsions are emulsions stabilized by solid particles. During emulsion preparation, said solid particles are positioned at the interface between the aqueous and oily phases.

Pickering emulsions according to an aspect of the present invention are oil-in-water emulsions, i.e. O/W, stabilized by a particular clay which is a modified natural phyllosilicate.

Beneficially, the aqueous phase of the Pickering emulsion is present in an amount ranging from 51% to 90%, such as 55% to 70% and for example 58% to 65% by weight relative to the total weight of the composition.

According to an embodiment, the modified natural phyllosilicate comprises a phyllosilicate selected from the group consisting of vermiculites and smectites and any combination thereof. In an embodiment, the phyllosilicate can be selected from the group consisting of montmorillonites, bentonites, nonytronites, beidellites, volkonskoites, hectorites, saponites, sauconites, sobockites, stevensites, svinfordites, such as sodium, potassium or calcium phyllosilicate, or mixtures thereof. In an embodiment, the phyllosilicate is selected from the group consisting of hectorite, montmorillonite, bentonite or mixtures thereof

According to an embodiment, the modified natural phyllosilicate comprises an organic compound selected from the group consisting of xanthan gum, guar gum, Tara gum or Carob gum, Acacia gum, carrageenan, alginate, chitosan, pectin, citric acid, tartaric acid, oxalic acid, succinic acid, malic acid, acetic acid, lactic acid, propionic acid, salicylic acid, glycosaminoglycans and any combination thereof. In an embodiment, the modified natural phyllosilicate comprises a bentonite modified with xanthan gum and citric acid.

According to another embodiment, the modified natural phyllosilicate comprises an organic compound selected from any organic compound known in the art of Pickering emulsions, such as those disclosed in French Patent No. FR 2,976,503.

In an embodiment, the modified phyllosilicate is present in an amount ranging from 3% to 20%, such as 4% to 10% and for example 5% to 8% by weight relative to the total weight of the composition.

According to an embodiment, the vegetable oil phase of the Pickering emulsion comprises at least one vegetable oil selected from the group consisting of by hemp oil, sunflower oil, rapeseed oil, olive oil, camelina oil, peanut oil, coconut oil, grapeseed oil, castor oil, argan oil, Djansang oil, desert date palm oil, nigella oil, prickly pear oil, macadamia oil, soybean oil, palm and palm kernel oil, Tamanu oil, sesame oil, linseed oil, walnut oil, hazelnut oil, baobab oil, passion fruit oil, Brazil nut oil, Hibiscus oil, Pumpkin seed oil, Luffa oil, Carapa oil, Evening primrose oil, Borage oil, Avocado oil, Almond oil, Sea buckthorn oil, Apricot kernel oil, Cherry kernel oil, Apple seed oil, Pomegranate oil, Jojoba oil, rose hip oil, plum oil, shea butter, cocoa butter, Kokum butter, mango butter, moabi butter, Karanja butter, Tucuma butter, Cupuaçu butter, Buriti butter, Murumuru butter, Kombo butter, Kpangnan butter, caprylic/capric acid triglycerides and any combination thereof.

Beneficially, the oil phase of the Pickering emulsion is present in an amount ranging from 5% to 40%, such as 10% to 30% and for example 15% to 25% by weight relative to the total weight of the composition.

### Composition

Beneficially, the composition according to the present invention comprises, in an acceptable medium, at least one cosmetic agent chosen from humectants, thickeners, texturizing agents, emulsifiers, dispersing agents, foaming agents, emollients, preservatives, colorants, plant extracts, plant fibers, minerals, pH correcting agents, active ingredients and fragrances.

Beneficially, the composition according to the present invention comprises from 0.001 to 20% by weight of at least one cosmetic agent relative to the total weight of the composition.

It will be appreciated that the person skilled in the art will take care to choose these cosmetic agents in such a way as not to alter the properties of the composition according to the invention.

The composition according to an aspect of the invention can be a massage composition, a skin care composition, and in particular a cleansing, protective, treatment or care cream for face, hands, feet or body, in particular the composition can be a day cream, night cream, make-up remover cream, foundation cream, sun protection cream; a fluid foundation, a cleansing milk, a protective or care body milk, an anti-sun milk; a lotion, gel or foam for skin care, such as a micellar cleansing lotion.

Beneficially, the process for preparing the composition according to an aspect of the invention comprises at least the following steps in this order: prepare the aqueous phase;

adding the organomodified phyllosilicate to the aqueous phase and mixing; add the vegetable oily phase to the mixture obtained and obtain a O/W
Pickering emulsion;
add a solution containing at least one hemp derivatives chosen from hemp flower oily macerate, cannabinoids, terpenes and flavonoids to the O/W Pickering emulsion.

It will be appreciated that the vegetable oil phase of the Pickering emulsion is different from the solution containing at least one hemp flower oily macerate.

It has been noted that the viscosities of hemp flower oil macerates are compatible with the process for preparing compositions according to the invention.

### Applications

The composition according to an aspect of the invention is beneficially intended for application to the skin.

It will be appreciated that aspects of the present invention also cover the use of the composition according to one or more embodiments the invention as a soothing, relaxing, anti-stress agent for the skin. It also covers a treatment method for the skin, comprising at least one step of applying the composition according to the invention to the skin.

In one or more embodiments, the composition according to the invention is also used for its anti-inflammatory action.

According to a particular aspect, the composition according to the invention is used to relieve inflammatory and ligament pains, insect bites and skin inflammations more particularly psoriasis.

The following examples are intended to illustrate the invention without limiting its scope.

Surprisingly, the combination of a hemp flower oily macerate and a Pickering emulsion containing an organomodified phyllosilicate results in a composition with significantly enhanced lipoxygenase enzymatic activity and IL-6 inhibition. In particular, formulation I1 exhibited more than 2.6× the basal lipoxygenase activity and over 40% IL-6 suppression, while neither the oily macerate alone nor the Pickering base alone (comparative examples C1 and C2) provided any such enhancement. Furthermore, a similar combination using kaolin (comparative C3) led to inhibition rather than stimulation. These results were unexpected and suggest a synergistic effect attributable to the specific selection and ratio of ingredients.

### EXAMPLES

### A. Macerates

### Example A1—Macerate 1

Table 1 lists the products used to prepare macerates.

TABLE 1

| Product | Quantity | Supplier |
|---|---|---|
| Organic hemp seed oil | 7.5 L | ABCD (France) |
| 1^{ère} cold pressing | | |
| Deflavored sunflower oil | 2.5 L | (Bouton D′or Bio) |
| | | France |
| Hemp Kompolti-Manicured | 5000 g | ABCD (France) |
| flowers | | |
| Chanvre Carmagnola-Manicured | 2000 g | ABCD (France) |
| flowers | | |
| Hemp Perugina-Manicured | 1000 g | ABCD (France) |
| flowers (greenhouse) | | |

The hemp flowers used are all dried, manicured and then ground a 250 micron cut-off.

They are then blended to form a mix of crushed hemp flowers.

### Preparation

The first reactor contained 2.5 L of deflavored sunflower oil and 2000 g of crushed flower mix. This mixture was then heated in a water bath at 95° C. for 1 hour to decarboxylate certain active components (conversion of CBA to CBD, CBGA to CBG and THCA to THC).

This “decarboxylated” oil is then filtered.

In a second reactor, 7.5 L of hemp seed oil and 6,000 g of crushed flower mix were introduced. This mixture was left to macerate for 3 weeks. During these 3 weeks, the macerate was stirred every day for 3 minutes.

The macerate obtained in the second reactor is then left to settle for 3 days, i.e. it is not stirred. It is then filtered through a coffee filter. The flowers remaining at the bottom of the settling tank are collected and pressed using an ECOLEA oil press.

Decanter oil, bottom press oil—from the second reactor—and sunflower oil—from the first reactor—are combined. The resulting oil is called Macerate 1.

HPLC analysis, using a Shimadzu DAD (diode array detector), of Macerate 1 determined the following contents recorded in Table 2:

TABLE 2

| Compounds | % mass |
|---|---|
| CBD | 0.188 |
| CBDA | 0.543 |
| THC | 0.024 |
| CBG | 0.096 |
| CBGA | 0.553 |

This gives a CBD contentₜₒₜₐₗ of 0.664% and a CBG contentₜₒₜₐₗ of 0.582%. To obtain these values, the acid forms (CBDA and CBGA) were multiplied by a factor of 0.878.

### Example A2—Macerate 2

Table 3 lists the products used to prepare the macerates.

TABLE 3

| Product | Quantity | Supplier |
|---|---|---|
| Organic hemp seed oil | 7.5 L | ABCD (France) |
| 1^{ère} cold pressing | | |
| Deflavored sunflower oil | 2.5 L | (Bouton D′or Bio) |
| | | France |
| Hemp Kompolti-Manicured | 3000 g | ABCD (France) |
| flowers | | |
| Chanvre Carmagnola-Manicured | 1000 g | ABCD (France) |
| flowers | | |
| Hemp Perugina-Manicured | 1000 g | ABCD (France) |
| flowers (greenhouse) | | |
| Kompolti trichome (powder from | 500 g | ABCD (France) |
| flowers ground to 250 um in olive | | |
| oil) | | |

The hemp flowers used are all dried, manicured and then ground to a 250 micron cut-off.

They are then blended to form a mix of crushed hemp flowers.

### Preparation

In a first reactor, 2.5 L of deflavored sunflower oil and 1250 g of crushed flower mix were introduced. This mixture was then heated over low heat, at 125° C., for 30 minutes to decarboxylate certain active components (transformation of CBA into CBD, CBGA into CBG and THCA into THC). This decarboxylated oil is then filtered.

In a second reactor, 7.5 L of hemp oil, 3750 g of crushed flower mix and 500 g of Kompolti Trichome were introduced. This mixture was left to macerate for 3 weeks. During these 3 weeks, the macerate is stirred every day for 3 minutes.

The macerate obtained in the second reactor is then left to settle for 3 days, i.e. it is not stirred. It is then filtered through a coffee filter.

The decantation oil from the second reactor is combined with the sunflower oil from the first reactor. The resulting oil is called Macerate 2.

HPLC analysis of Macerate 2, using a Thermo Scientific Vanquish DAD, determined the following contents, recorded in Table 4:

TABLE 4

| Compounds | % mass |
|---|---|
| CBD | 2.025 |
| CBDA | 0.596 |
| THC | 0.032 |
| CBG | 0.029 |
| CBGA | 0.239 |

This gives a CBD content total of 2.548% and a CBG content total of 0.239%. To obtain these values, the acid forms (CBDA and CBGA) were multiplied by a factor of 0.878.

### Example A3—Macerate 3

Table 5 lists the products used to prepare the macerates.

TABLE 5

| Product | Quantity | Supplier |
|---|---|---|
| Organic hemp seed oil | 10.0 L | ABCD (France) |
| 1^{ère} cold pressing | | |
| Hemp Kompolti-Manicured | 5000 g | ABCD (France) |
| flowers | | |
| Chanvre Carmagnola-Manicured | 1000 g | ABCD (France) |
| flowers | | |

The hemp flowers used are all dried, manicured and then ground to a 250 micron cut-off.

2500 g of Kompolti flowers and 1000 g of Carmagnola flowers are crushed and blended to form a mix of crushed hemp flowers.

### Preparation

The remaining 2500 g of Kompolti flowers are heated in an oven at 120° C. for 20 minutes. This treatment decarboxylates the Kompolti flowers.

The 10.0 L of hemp oil, 2500 g of decarboxylated Kompolti flowers and 3500 g of crushed hemp flower mix were introduced into a reactor. This mixture is then left to macerate for 3 weeks. During these 3 weeks, the macerate is stirred every day for 3 minutes.

The resulting macerate is then filtered through a coffee filter.

The oil obtained is called Macerate 3.

HPLC analysis Macerate 3, using a Thermo Scientific Vanquish DAD, yielded the following results, shown in Table 6.

TABLE 6

| Compounds | % mass |
|---|---|
| CBD | 1.044 |
| CBDA | 0.521 |
| THC | 0.033 |
| CBG | 0.030 |
| CBGA | 0.084 |

This gives a CBD contentₜₒₜₐₗ of 1.502% and a CBG contentₜₒₜₐₗ of 0.103%. To obtain these values, the acid forms (CBDA and CBGA) were multiplied by a factor of 0.878.

It has been verified that Macerates 1, 2 and 3 are similar in terms of their targeted soothing properties

### B. Compositions Tested

1—C1 comparative composition

Comparative composition C1 corresponds to Macerate M1.

2—C2 comparative composition

Comparative composition C2 comprises a O/W Pickering emulsion containing a modified phyllosilicate clay (without hemp oil macerate).

Table 7 lists the products used prepare composition C2.

TABLE 7

| Phase | Trade name | % | INCI |
|---|---|---|---|
| A | Water | 73.70 | Aqua |
| B | Georgard Ultra | 1.00 | Gluconolactone & Sodium |
| | marketed by | | benzoate & Calcium gluconate |
| | Lonza | | |
| B | Sodium | 0.30 | Sodium benzoate |
| | Benzoate | | |
| C | Frametime CXG | 4.50 | Bentonite & xanthan gum & |
| | marketed by | | Sodium stearoyl glutamate & citric |
| | Ephyla | | acid |
| C | FF xanthan gum | 0.50 | xanthan gum |
| | marketed by | | |
| | Jungbunzlauer | | |
| D | Refined | 20.00 | Helianthus annuus seed oil |
| | sunflower oil | | |
| | marketed by | | |
| | CAUVIN | | |
| Total | | 100 | |

C2 composition preparation

Phases A and B are mixed to form an aqueous phase, then phase C is added and the whole is mixed. Finally, oily phase D is added and the whole is mixed. Once the Pickering emulsion has been obtained, the pH is adjusted to 5.00.

3—C3 composition

Comparative composition C3 comprises a macerate and a O/W Pickering emulsion containing a kaolin.

Table 8 lists the products used to prepare composition C3.

TABLE 8

| Phase | Trade name | % | INCI |
|---|---|---|---|
| A | Water | 70.30 | Aqua |
| B | Georgard Ultra | 1.00 | Gluconolactone & Sodium |
| | marketed by | | benzoate & Calcium gluconate |
| | Lonza | | |
| B | Sodium | 0.30 | Sodium benzoate |
| | Benzoate | | |
| C | White clay | 4.50 | Kaolin |
| | marketed by | | |
| | COOPER | | |
| C | FF xanthan gum | 0.50 | xanthan gum |
| | marketed by | | |
| | Jungbunzlauer | | |
| D | Refined | 20.00 | Helianthus annuus seed oil |
| | sunflower oil | | |
| | marketed by | | |
| | Cauvin | | |
| E | Macerate 1 | 3.40 | |
| Total | | 100 | |

Preparing the C3 composition:

Phases A and B have been mixed to form an aqueous phase, then phase C has been added and the whole has been mixed. Finally, oily phase D is added and the whole is mixed.

Once the Pickering emulsion has been obtained, phase E, that is Macerate 1, is added and mixed. Finally, the pH adjusted to 5.00.

4—Composition I1 according to invention

Composition I1, according to the invention, comprises a macerate and a O/W Pickering emulsion containing a modified phyllosilicate clay.

Table 9 lists the products used to prepare composition I1.

TABLE 9

| Phase | Trade name | % | INCI |
|---|---|---|---|
| A | Water | qsp | Aqua |
| B | Frametime CX | 6.00 | Bentonite & xanthan |
| | marketed by | | gum & citric acid |
| | Ephyla | | |
| B | Amisoft Hs11p | 0.30 | Sodium stearoyl glutamate |
| B | FF xanthan gum | 0.50 | Xanthan gum |
| | marketed by | | |
| | Jungbunzlauer | | |
| C | Cetosaryl | 4.00 | Cetearyl alcohol |
| | alcohol 50/50 | | |
| C | Ephyster MCR | 20.00 | Brassica napus extract |
| C | HTR1 marketed | 1.50 | Helianthus annuus seed oil & |
| | by EPHYLA | | Protium heptaphyllum resin |
| C | LD Sepicide | 1.00 | Phenoxyethanol |
| D | Macerat 1 | 3.40 | |
| total | | 100.00 | |

Preparation of composition I1

Phases A and B have been mixed to form an aqueous phase, then phase C is added and the whole is blended

Once the Pickering emulsion has been obtained, phase D, i.e. Macerate 1, is added and mixed. Finally, the pH is adjusted to 5.00.

### C. Activity on a Skin Enzyme in an In Vitro Model

Tests were carried out in vitro, on the key enzyme in the arachidonic cascade: lipoxygenase, an enzyme present in the skin's epidermis.

This study is based on the influence of cosmetic products on the activity of the enzyme Lipoxygenase, so as to be able to compare and classify the influence of these products on this enzymatic activity. This study aims to illustrate the impact of topical galenics on enzymatic activity in the skin.

The lipoxygenase enzyme activity of several compositions was measured in an in vitro cell model using a kit.

### Materials and Reagents

The test kit used is the Lipoxygenase Inhibitor Screening Assay Kit sold by CAYMAN/INTERCHIM.

Table 10 lists the kit components

TABLE 10

| | |
|---|---|
| TAMPON | CAYMAN/INTERCHIM |
| TRIS-HCL | |
| CHROMOGEN 1 = development | CAYMAN/INTERCHIM |
| reagent 1 | |
| CHROMOGEN 2 = development | CAYMAN/INTERCHIM |
| reagent 2 | |
| ENZYME: 15-LIPOXYGENASE | CAYMAN/INTERCHIM |
| ARACHIDONIC CID | CAYMAN/INTERCHIM |
| POTASSIUM HYDROXIDE | CAYMAN/INTERCHIM |
| 0.1M | |
| NDGA INHIBITOR | CAYMAN/INTERCHIM |

### Spectrometer

Measurements were carried out using a POLARstar Omega spectrometer from BMG LABTECH

### Reagent Preparation

1. R1: Lipoxygenase inhibitor screening assay buffer. 3 ml of concentrated TAMPON have been diluted with 27 ml of HPLC-grade water.

Test Buffer R1 (0.1 M Tris-HCl, pH 7.4) should be used to dilute samples and ENZYME: 15-LIPOXYGENASE (standard 15-LO) prior to assay. When stored at 4° C., this R1 test buffer is stable for at least two months.

R2 Chromogen

Chromogen R2 has been prepared before use by mixing equal volumes of CHROMOGEN 1 and CHROMOGEN 2 in a test tube and vortexing. The volume of Chromogen R2 to be prepared depends on the number of wells to be analyzed (100 μl for each well). Chromogen R2 should be used within the hour.

R3:15-lipoxygenase standard

10 μl of ENZYME: 15-LIPOXYGENASE has been transferred into a vial and dilute with 990 μl of R1 before use, keep on ice and use within one hour.

R4 Arachidonic acid (substrate)

The vial supplied in the kit contains a solution of arachidonic acid (ARACHIDONIC ACID) in ethanol and should be stored at −20° C. 25 μl of ARACHIDONIC ACID have been transferred to another vial, 25 μl of potassium hydroxide solution have been added, vortexed and diluted with 950 μl of HPLC-grade water to give 1 mM solution R4. An aliquot of 10 μl will give a reaction concentration of 91 μM in the wells.

Kit contains one vial of ready-to-use 0.1 M potassium hydroxide (KOH)

R5—NDGA positive control inhibitor

The kit contains one vial of 550 nmol non-selective nordihydroguaiaretic acid (NDGA) lipoxygenase inhibitor
Resuspend in 500 μl of R1 to make a 1.1 mm stock. The addition of 10 μl to the assay gives a final result of 100 M inhibitor concentration in the well
Each well has a final volume of 210 μl.

### Test Execution

1. Sample blank wells (Sample blank):

add 90 μl of solution R1 and 10 μl of triplicate test material

2. Positive control well (basal activity=Control +):

add 90 μl of R3 solution and 10 μl of solution R1 in triplicate.

3. Well Test product:

add 90 μl of R3 solution and 10 μl of test product in triplicate.

4. Inhibitor well (Control−)

add 90 μl of R3 solution 10 μl of solution R5 in triplicate;
5. Incubate for five minutes at room temperature.

6. Initiate reaction by adding 10 μl of R4 solution to all wells. Place the 96-well plate on a shaker for at least ten minutes.

7. Add 100 μl of R2 solution to each well to develop the reaction. Cover with a plate cover and place the 96-well plate on a shaker for five minutes.

8. Remove the lid and read the absorbance at 500 nm using a plate reader.

### Reaction Principle

A buffered solution of lipoxygenase (R3) reacts with a specific substrate, arachidonic acid (R4), and converts it to a compound that binds to a chromogen (R2), with stirring at room temperature. Lipoxygenase activity can thus be assessed by measuring absorbance at 500 nm.

The sample or reference product: NDGA inhibitor (R5) is brought into contact with the lipoxygenase solution (R3) together with the enzyme substrate (R4). The substrate (R4) transformed by the enzyme is stained with chromogen R2 by agitation at room temperature. Lipoxygenase activity in the various wells is then assessed by measuring absorbance at 500 nm

Modulation of this activity is expressed as a percentage inhibition or activation of lipoxygenase activity relative to the basal level of activity in the absence of sample or reference product, i.e. only in the presence of the enzyme substrate (arachidonic acid).

### Incubation Protocol

A solution of lipoxygenase enzyme is incubated in its substrate, arachidonic acid, for 10 minutes, in the absence or presence of the reference inhibitor and the test sample. The chromogen is then incorporated before incubation for 35 minutes at room temperature on a Incu-Shaker Mini orbital shaker marketed by Benchmark company.

### Effect Assessment

At the end of the incubation period, the activity of the lipoxygenase enzyme with and without test or reference product was assessed by measuring the absorbance of the reaction media at 500 nm.

For each tested concentration, the modulation of lipoxygenase enzyme activity by the test product is calculated according to the following formula

Percentage of modulation of lipoxygenase enzyme activity=100×[(DO test product−DO lipoxygenase alone (blank))/DO lipoxygenase alone]

If the result is negative, the percentage is expressed as enzyme inhibition.

Results in terms of OD average, OD average minus OD blank, % enzyme activity are shown in Table 11

TABLE 11

| | | OD | Enzyme |
|---|---|---|---|
| | OD | average- | activity |
| Sample | average | OD blank | (%) |
| Blank | 0.221 | 0.000 | 0.00 |
| T+ | 0.436 | 0.215 | 100.00 |
| T− | 0.187 | −0.034 | 0.00 |
| C1 | 0.812 | 0.217 | 101.24 |
| C2 | 1.653 | −0.073 | 0.00 |
| C3 | 3.845 | 0.054 | 25.00 |
| I1 | 2.437 | 0.578 | 269.10 |

The level of significance between the “control” and the “reference product” was assessed using a Student's t-test (*: p<0.05).

The level of significance between the “control” and each “test compound” was assessed independently for each composition by a one-way analysis of variance (ANOVA) followed by a Holm-Sidak test (*: p<0.05).

It will be appreciated that when the macerate and the organomodified phyllosilicate are within the ranges disclosed herein, the synergistic and dramatically improved enzymatic activation occur. Deviations from the disclosed ranges-either by using different concentrations or alternate clays-fail to deliver this effect, indicating the relevance of the claimed concentration ranges.

### Conclusions

The composition according to invention I1 has an enzymatic activity of 269% (i.e. +169% compared with the normal basal activity of 100%), whereas the comparative composition C1 of hemp oily macerate alone has an enzymatic activity of 101%, and the comparative composition C2 comprising Pickering emulsion alone has an enzymatic activity of 0%.

It should also be noted that the comparative C3 composition comprising hemp oily macerate and a kaolin-based Pickering emulsion has an enzymatic activity of 25% only.

The composition according to an aspect of the invention has a synergistic effect on the studied enzyme activity, which is not observed when a kaolin-based Pickering emulsion is used.

Surprisingly and unexpectedly, composition I1 strongly stimulates enzymatic activity, while composition C1, i.e. Macerate 1, shows no modulation of the basal rate, and composition C2 (which forms the backbone of the composition according to the invention) without Macerate 1, on the contrary, totally inhibits enzymatic activity. Equally surprisingly, composition C3 comprising a Pickering emulsion made from kaolin shows an inhibition of the order of 75% of enzyme activity compared with the basal (=normal) rate of enzyme activity.

Macerate 1 (C1) alone does not modulate basal enzyme activity, the pickering galenic (C2) has an inhibitory effect on enzyme activity, while the composition according to the invention (I1) stimulates it.

### D—Measuring the Relaxing or De-stressing Effect on Human Skin Explants by Measuring IL-6 (Interleukin 6 Secretion).

The skin, and in particular the epidermis, secretes a basal level of cellular mediator that reflects both intercellular stress and environmental stress (it is a form of expression of the reactivity of our epidermis in contact with its external environment).

This study is based on the influence of the composition according to invention I1 on the ability to modulate Interleukin 6 production.

The desired effect is an inhibitory one, which translates into a soothing, relaxing or anti-stress effect on the epidermis in contact with the external environment.

### Principle of the Study

For this study, normal fresh human skin explants are exposed to the test samples for a contact time of 6 hours.

At the end of this contact time, the explants were dissected and suspended in saline. The IL-6 concentration of the explants was determined by Elisa assay using an Enzo IL-6 kit.

### Test System

For this study, human skin explants are exposed to the samples to be tested before being placed on Franz cells; placement on Franz cells induces an expected environmental stress as the skin is pinched and/or clipped onto the edges of the Franz cell.

Skin explant/Franz cell systems are maintained for 6 h at 32° C.±1° C. After incubation, the explants were rinsed, dissected and preserved in saline before being subjected to a 30-minute ultrasound cycle. The explants are then centrifuged for 15 minutes at 5000 rpm and 4° C., and the supernatants are stored and used for interleukin 6 assay.

Skin explant supernatants are used for interleukin 6 assays using the Enzo IL-6 (human) high sensitivity ELISA Kit. This kit provides a convenient assay for interleukin-6, enabling the modulatory effect of active ingredients or topical products to be assessed. The kit implements an enzyme-linked immunosorbent assay in which samples are brought into contact with monoclonal antibodies specific to interleukin 6 (IL-6) fixed on a microplate. After rinsing, a biotinylated anti-interleukin-6 antibody is added and binds specifically. A peroxidase solution is added, binds to the biotinylated antibody and then reacts with the addition of substrate to form a blue stain. The reaction is stopped and a yellow coloration is formed, the intensity of which is proportional to the concentration of interleukin-6. At the same time, an IL-6 calibration curve is produced and used to determine the concentration of IL-6 in skin explant supernatants.

In this study, each condition and/or sample tested was analyzed in triplicate with explants and for IL-6 assay.

### Results

### Standard Range

For this study, an interleukin 6 calibration range from 0.0 to 50.0 pg/mL was performed. The absorbances of the interleukin 6 standard range at 450 nm are shown in Table 12.

TABLE 12
Interleukin 6 range (pg/mL)

| | Blank | NSB | 0.0 | 0.5 | 1.0 | 5.0 | 10.0 | 50.0 |
|---|---|---|---|---|---|---|---|---|
| OD | 0.000 | 0.096 | 0.572 | 0.107 | 0.266 | 1.012 | 0.949 | 2.377 |
| 450 nm | | | | | | | | |
| OD | — | 0.000 | 0.476 | 0.01 | 0.170 | 0.916 | 0.853 | 2.281 |
| 450 nm- | | | | | | | | |
| NSB | | | | | | | | |

NSB: saline solution.

Between 0.5 and 5.0 pg/mL IL-6, the calibration curve is linear. The equation of this line a corresponds to y=0.1954x−0.0578

### Sample Measurement

Two controls were created for this study: a “basal” control for basal stress measurement and a solvent control, representative of the stress caused by the method and solvent used.

Absorbance (OD) results at 450 nm for controls and samples are shown in Tables 13 and 14, respectively, below.

TABLE 13
OD 450 nm of controls

| | Basal control | | | Solvent indicator | | |
|---|---|---|---|---|---|---|
| | Repli- | Repli- | Repli- | Repli- | Repli- | Repli- |
| | cate | cate | cate | cate | cate | cate |
| | 1 | 2 | 3 | 1 | 2 | 3 |
| OD | 0.711 | 1.377 | 1.086 | 0.306 | 0.710 | 0.447 |
| 450 nm | | | | | | |
| OD | 0.615 | 1.281 | 0.990 | 0.210 | 0.614 | 0.351 |
| 450 nm- | | | | | | |
| NSB | | | | | | |

TABLE 14
OD 450 nm of samples of composition I1

| | Replicate 1 | Replicate 2 |
|---|---|---|
| OD | 0.712 | 0.708 |
| 450 nm | | |
| OD | 0.616 | 0.612 |
| 450 nm- | | |
| NSB | | |

### IL-6 Concentrations

Control and sample IL-6 concentrations were determined using the calibration curve equation and are presented in Tables 15 and 16, respectively, below:

TABLE 15
OD 450 nm of controls

| | Basal control | | | Solvent indicator | | |
|---|---|---|---|---|---|---|
| | Repli- | Repli- | Repli- | Repli- | Repli- | Repli- |
| | cate | cate | cate | cate | cate | cate |
| | 1 | 2 | 3 | 1 | 2 | 3 |
| Concen- | 3.44* | 6.85 | 5.36 | 1.37 | 3.44* | 2.09 |
| tration | | | | | | |
| in IL-6 | | | | | | |
| (pg/mL) | | | | | | |

| | | |
|---|---|---|
| Average | 6.106 | 1.729 |
| Standard | 1.055 | 0.510 |
| deviation | | |

*values excluded as statistically outliers, not interpretable

TABLE 16
OD 450 nm of samples of composition I1

| | Replicate 1 | Replicate 2 |
|---|---|---|
| IL-6 | 3.45 | 3.43 |
| concentration | | |
| (pg/mL) | | |
| Average | 3.438 | |
| Standard | 0.014 | |
| deviation | | |

Under study conditions, the IL-6 concentration of the basal stress control is 6.106 pg/mL and that of the solvent-induced stress control is 1.729 pg/mL.

The mean IL-6 concentration of explants exposed to composition according to invention I1 (0.008 g) was 3.438 pg/mL, i.e. 43.7% response inhibition compared with the basal control.

### Conclusion:

Explants exposed to the sample “Composition according to invention I1” have an inhibited response compared with the basal control; at a dose of 0.008 g, the product tested inhibits the IL-6 response by 43.7%; “Composition according to invention I1” has a very significant soothing, relaxing or anti-stress effect on this skin explant and more particularly on the epidermis exposed to environmental stress.

The composition according to an aspect of the invention has an inhibitory action on the arachidonic cascade (LIPOX) and IL-6, and therefore has an anti-inflammatory action. The composition according to an aspect of the invention has been used to relieve inflammatory and ligament pain, insect bites and skin inflammation, particularly psoriasis.

Unlike prior art compositions (e.g., WO2016/123475), which address formulation challenges of Cannabis oil, the present invention demonstrates a novel and non-obvious synergy between hemp derivatives and a Pickering emulsion using an organomodified phyllosilicate. The disclosed compositions not only solve viscosity and stability issues but also yield unexpectedly superior biological effects. These effects are not observed in conventional formulations or when using kaolin-based Pickering systems.

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

The articles “a” and “an” may be employed in connection with various elements and components, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, components, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components and elements set forth in the foregoing description. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description and drawings are by way of example only.

### BIBLIOGRAPHY

1. Andre C M, Hausman J F, Guerriero G. Cannabis sativa: The plant of the thousand and one molecules. Front Plant Sci. 2016; 7:19.
2. LaVigne J E, Hecksel R, Keresztes A, Streicher J M. Cannabis Sativa terpenes are cannbimetic and selectively enhance cannabinoid activity. Nature Scientific Reports. 2021; 11:83232.
3. Russo E B. The Case for the Entourage Effect and Conventional Breeding of Clinical Cannabis: No “Strain,” No Gain. Frontiers in Plant Science. Perspective. 2019; 9:1-8.

1. A composition comprising a solution containing at least one hemp derivative and a O/W Pickering emulsion, said at least one hemp derivative being selected from the group consisting of hemp flower oily macerates, cannabinoids, terpenes and flavonoids and any combination thereof and the O/W Pickering emulsion containing an aqueous phase, a vegetable oily phase and an organomodified phyllosilicate.

2. The composition according to claim 1, wherein the solution contains at least one oily hemp flower macerate and a O/W Pickering emulsion containing the aqueous phase, the vegetable oil phase and the organomodified phyllosilicate.

3. The composition according to claim 2, wherein the solution containing at least one oily macerate of hemp flowers comprises at least CBD in a quantity ranging from 0.005% to 24.000% by weight relative to the total weight of the solution; CBDA in a quantity ranging from 0.005% to 24.000% by weight relative to the total weight of the solution; THC in an amount ranging from 0.001% to 30.000% by weight based on the total weight of the solution; CBG in an amount ranging from 0.001% to 22.000% by weight based on the total weight of the solution and CBGA in an amount ranging from 0.001% to 22.000% by weight based on the total weight of the solution.

4. The composition according to claim 3, wherein the solution containing at least one oily macerate of hemp flowers comprises at least CBD in a quantity ranging from 0.01% to 5.000% by weight relative to the total weight of the solution; CBDA in a quantity ranging from 0.010% to 5.000% by weight relative to the total weight of the solution; THC in an amount ranging from 0.010% to 1.000% by weight based on the total weight of the solution; CBG in an amount ranging from 0.010% to 3.000% by weight based on the total weight of the solution and CBGA in an amount ranging from 0.010% to 3.000% by weight based on the total weight of the solution.

5. The composition according to claim 2, wherein the solution containing at least one oily macerate of hemp flowers is present in a content ranging from 0.1 to 25%, by weight relative to the total weight of the composition.

6. The composition according to claim 5, wherein the at least one oily macerate of hemp flowers is present in a content ranging from 1% to 5%, by weight relative to the total weight of the composition.

7. The composition according to claim 1, wherein the organomodified phyllosilicate is present in an amount ranging from 3% to 20%, by weight relative to the total weight of the composition.

8. The composition according to claim 1, wherein the organomodified phyllosilicate is present in an amount ranging from 4% to 10% by weight relative to the total weight of the composition.

9. The composition according to claim 1, wherein the aqueous phase of the O/W Pickering emulsion is present in an amount ranging from 51% to 90%, by weight relative to the total weight of the composition.

10. The composition according to claim 9, wherein the aqueous phase of the O/W Pickering emulsion is present in an amount ranging from 55% to 70% by weight relative to the total weight of the composition.

11. The composition according to claim 1, wherein the vegetable oily phase of the O/W Pickering emulsion comprises at least one vegetable oil selected from the group consisting of hemp oil, sunflower oil, rapeseed oil, olive oil, camelina oil, peanut oil, coconut oil, grapeseed oil, castor oil, argan oil, Djansang oil, desert date palm oil, nigella oil, prickly pear oil, macadamia oil, soybean oil, palm and palm kernel oil, Tamanu oil, sesame oil, linseed oil, walnut oil, hazelnut oil, baobab oil, passion fruit oil, Brazil nut oil, Hibiscus oil, Pumpkin seed oil, Luffa oil, Carapa oil, Evening primrose oil, Borage oil, Avocado oil, Almond oil, Sea buckthorn oil, Apricot kernel oil, Cherry kernel oil, Apple seed oil, Pomegranate oil, Jojoba oil, rose hip oil, plum oil, shea butter, cocoa butter, Kokum butter, mango butter, moabi butter, Karanja butter, Tucuma butter, Cupuaçu butter, Buriti butter, Murumuru butter, Kombo butter, Kpangnan butter, caprylic/capric acid triglycerides and any proper combination thereof.

12. The composition according to claim 1, wherein the oily phase of the O/W Pickering emulsion is present in an amount ranging from 5% to 40%, by weight relative to the total weight of the composition.

13. The composition according to claim 12, wherein the oily phase of the O/W Pickering emulsion is present in an amount ranging from 10% to 30% by weight relative to the total weight of the composition.

14. The composition according to claim 1, comprising, in an acceptable medium, at least one cosmetic agent chosen from humectants, thickeners, texturizing agents, emulsifiers, dispersing agents, foaming agents, emollients, preservatives, colorants, plant extracts, plant fibers, minerals, pH-correcting agents, active ingredients and fragrances.

15. The composition according to claim 1, wherein the composition is formulated to be applied to the skin.

16. A process for preparing the composition according to claim 1, comprising at least the following steps in this order:
preparing the aqueous phase;
adding the organomodified phyllosilicate to the aqueous phase and mixing;
adding the vegetable oily phase to the mixture obtained and obtain a O/W Pickering emulsion;
adding a solution containing at least one hemp derivatives chosen from hemp flower oily macerate, cannabinoids, terpenes and flavonoids to the O/W Pickering emulsion.

17. A method comprising applying the composition according to claim 1 as a soothing, relaxing, anti-stress agent for the skin.

18. A process for the treatment of the skin, comprising at least one step of applying to the skin the composition according to claim 1.
